(19) United States
(12) Patent Application Publication
(10) Pub. No.: US 2025/0331744 A1
(43) Pub. Date: Oct. 30, 2025

(54) MEASURING INSTRUMENT

(71) Applicant: Sharp Semiconductor Innovation Corporation, Tenri City (JP)

(72) Inventors: Son Nam HOANG, Tenri City (JP); Daisuke FUNAO, Tenri City (JP); Qichen WANG, Tenri City (JP); Sota HIDA, Tenri City (JP); Yuta MOCHIZUKI, Tenri City (JP)

(21) Appl. No.: 19/189,532

(22) Filed: Apr. 25, 2025

(30) Foreign Application Priority Data
Apr. 26, 2024 (JP) 2024-072906

(51) Int. Cl. A61B 5/1455; H04N 23/10

(52) U.S. Cl. CPC A61B 5/14552; H04N 23/10

(57) ABSTRACT

A measuring instrument including: a camera that captures an image of a living body at a distance from the living body, the camera including an imaging element including: a group of first pixels that exhibit a peak sensitivity wavelength of greater than or equal to 620 nm and less than or equal to 740 nm; and a group of second pixels including a group of pixels of at least one color, the second pixels exhibiting a peak sensitivity wavelength of either less than or equal to 600 nm or greater than or equal to 760 nm, the group of first pixels having a total light-receiving area that is larger than a total light-receiving area of a group of pixels of each color; and a processing unit that processes a signal representing quantities of light received by the group of first pixels and the group of second pixels.

### CROSS-REFERENCE TO RELATED APPLICATION

The present application claims priority from Japanese Application JP2024-072906, the content of which is hereby incorporated by reference into this application.

### BACKGROUND OF THE INVENTION

### 1. Field of the Invention

The present disclosure relates to a measuring instrument.

### 2. Description of the Related Art

PCT International Application Publication No. WO2017/104056 discloses a biological information measuring instrument. In this biological information measuring instrument, first pixels are sensitive to light that is in the wavelength range of from 530 nm to 590 nm that falls in the sensitivity region of a heartbeat component. In addition, second pixels receive light that is in the wavelength ranges of from 500 nm to 530 nm and from 590 nm to 620 nm, both of which fall outside the sensitivity region of the heartbeat component. In addition, first time-series data and second time-series data are generated respectively for an average value of a first luminance that is a signal value of an electric signal contained in a first image capturing signal generated by the first pixels and for an average value of a second luminance that is a signal value of an electric signal contained in a second image capturing signal generated by the second pixels In addition, the heartbeat component and an illumination variable component are separated from the first time-series data and the second time-series data. Hence, the heartbeat component and the illumination variable component can be separated with high precision (paragraphs 0014, 0019, 0020, 0025, 0026, 0029, and 0030).

### SUMMARY OF THE INVENTION

In the biological information measuring instrument disclosed in Patent Literature 1, the second pixels are sensitive to light that is in a narrow wavelength range and for this reason, exhibit low sensitivity. Therefore, the second time-series data is easily affected by noise. Therefore, the separation of the heartbeat component and the illumination variable component are affected by noise.

The present disclosure, in an aspect thereof, has been made in view of this problem. The present disclosure, in an aspect thereof, has an object to provide, for example, a measuring instrument that enables restraining the influence of noise on the measurement of biological information.

The present disclosure, in an aspect thereof, is directed to a measuring instrument including: a camera that captures an image of a living body at a distance from the living body, the camera including an imaging element including: a group of first pixels that exhibit a peak sensitivity wavelength of greater than or equal to 620 nm and less than or equal to 740 nm; and a group of second pixels including a group of pixels of at least one color, the second pixels exhibiting a peak sensitivity wavelength of either less than or equal to 600 nm or greater than or equal to 760 nm, the group of first pixels having a total light-receiving area that is larger than a total light-receiving area of a group of pixels of each of the at least one color; and a processing unit that acquires biological information of the living body by processing a signal representing quantities of light received by the group of first pixels and the group of second pixels.

### BRIEF DESCRIPTION OF THE DRAWINGS

FIG. 1 is a block diagram of a measuring instrument in accordance with Embodiment 1.

FIG. 2 is a schematic cross-sectional view of a camera included in the measuring instrument in accordance with Embodiment 1.

FIG. 3 is a schematic plan view of a group of pixels included in the measuring instrument in accordance with Embodiment 1.

FIG. 4 is a diagram illustrating the content of a process of acquiring a volume pulse wave and image data performed by a processing unit included in the measuring instrument in accordance with Embodiment 1.

FIG. 5 is a graph representing an example of the spectral sensitivities of pixels included in the measuring instrument in accordance with Embodiment 1.

FIG. 6 is a schematic plan view of a basic unit included in a measuring instrument in accordance with a first variation example of Embodiment 1.

FIG. 7 is a schematic plan view of a basic unit included in a measuring instrument in accordance with a second variation example of Embodiment 1.

FIG. 8 is a schematic plan view of a basic unit included in a measuring instrument in accordance with a third variation example of Embodiment 1.

FIG. 9 is a schematic plan view of a basic unit included in a measuring instrument in accordance with a fourth variation example of Embodiment 1.

FIG. 10 is a schematic plan view of a basic unit included in a measuring instrument in accordance with a fifth variation example of Embodiment 1.

FIG. 11 is a schematic plan view of a basic unit included in a measuring instrument in accordance with a sixth variation example of Embodiment 1.

FIG. 12 is a schematic plan view of a basic unit included in a measuring instrument in accordance with a seventh variation example of Embodiment 1.

FIG. 13 is a schematic plan view of a basic unit included in a measuring instrument in accordance with an eighth variation example of Embodiment 1.

FIG. 14 is a diagram illustrating the content of a process of acquiring a volume pulse wave and image data performed by a processing unit included in the measuring instrument in accordance with the eighth variation example of Embodiment 1.

### DETAILED DESCRIPTION OF THE INVENTION

The following will describe embodiments of the present disclosure with reference to drawings. Identical and equivalent elements in the drawings are denoted by the same reference numerals, and description thereof is not repeated.

### 1 Embodiment 1

### 1.1 Measuring Instrument

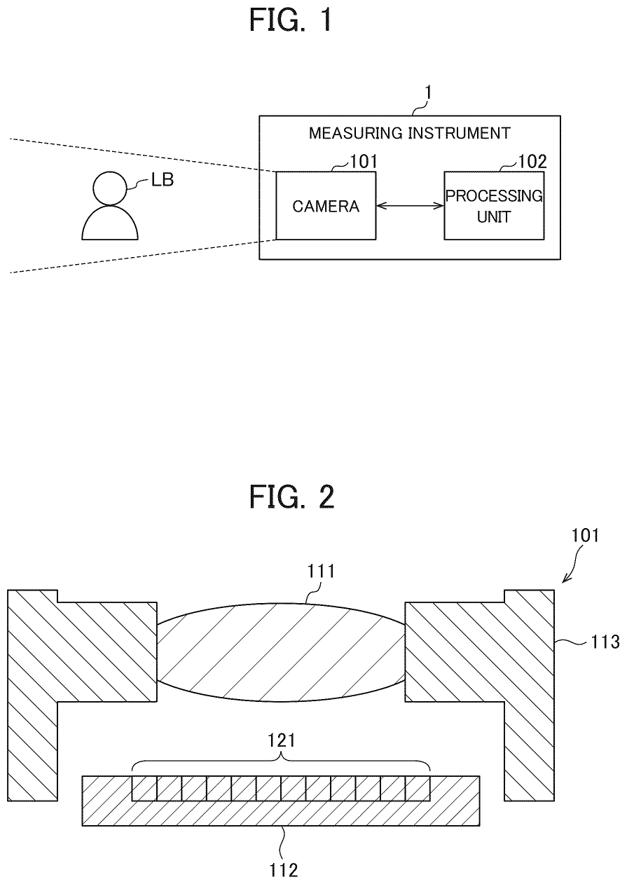

FIG. 1 is a block diagram of a measuring instrument in accordance with Embodiment 1.

A measuring instrument 1 in accordance with Embodiment 1 shown in FIG. 1 measures biological information of a living body LB. In doing so, the measuring instrument 1 generates a signal in accordance with the light reflected off the living body LB by capturing an image of the living body LB and acquires biological information from the generated signal. Hence, the measuring instrument 1 measures biological information in a contactless manner. The measured biological information may include a volume pulse wave as well as, for example, a heart rate, a blood pressure, a respiration rate, and a blood oxygen saturation level calculated from the volume pulse wave.

The living body LB is the body of a living organism. This living organism has a cardiovascular system for circulating blood containing oxidized hemoglobin and reduced hemoglobin. The living organism is, for example, a human.

The measured volume pulse wave is caused by variations of the intensity of the light reflected off the living body LB, the variations being in turn caused by repetition of alternate expansion and contraction of blood vessels in which blood flows.

The variations of the intensity of the reflected light reflect the expansion and contraction of blood vessels in a thin area along the surface of the skin of the living body LB. Therefore, the variations of the intensity of the reflected light are as small as approximately a few tenths of a percent. For example, when the intensity of reflected light is represented by a numerical value with a bit length of 12 bits, the variations of the intensity of the reflected light are as small as approximately 10 LSB even if the intensity of the reflected light is approximately 1,000 LSB. Therefore, the measured volume pulse wave has a small amplitude. Therefore, the volume pulse wave measured in a contactless manner is generally easily affected by, for example, body movement artifacts of the living body LB, variations of environmental light, and noise on signals representing the quantities of the light received by pixels. Therefore, it is generally difficult to measure a volume pulse wave with high precision in a contactless manner. The measuring instrument 1 can overcome this problem, thereby measuring a volume pulse wave with high precision in a contactless manner.

Referring to FIG. 1, the measuring instrument 1 includes a camera 101 and a processing unit 102.

The camera 101 captures an image of the living body LB at a distance from the living body LB and outputs a signal in accordance with reflected light.

The processing unit 102 controls the camera 101. The processing unit 102 acquires biological information from the outputted signal. The processing unit 102 includes a processor, a memory, and peripheral circuitry. The processor executes a program stored in the memory to cause the processor, the memory, and the peripheral circuitry to function as the processing unit 102. The process performed by the processing unit 102 may be either entirely or partially performed by a dedicated electronic circuit.

The camera 101 captures an image of the skin of the living body LB, preferably an image of the skin of the face of the living body LB. If the camera 101 captures an image of the skin of the face of the living body LB, the biological information can be acquired from a signal that is in accordance with the light reflected off the skin which has a large area and below which there exist many blood vessels. Therefore, the biological information can be easily acquired.

### 1.2 Camera

FIG. 2 is a schematic cross-sectional view of a camera included in the measuring instrument in accordance with Embodiment 1.

Referring to FIG. 2, the camera 101 includes a lens 111, an imaging element 112, and a support member 113.

The lens 111 guides the light reflected off the living body LB to the imaging element 112. The lens 111 focuses the reflected light onto the imaging element 112 to form an image of the living body LB on the imaging element 112.

Referring to FIG. 2, the imaging element 112 includes a group of pixels 121. The group of pixels 121 includes a plurality of pixels arranged in a matrix in a light-receiving face that is perpendicular to the optical axis of the lens 111. The group of pixels 121 may include a plurality of pixels arranged in a non-matrix manner. The imaging element 112 outputs a signal representing the quantity of the light received by the group of pixels 121. The imaging element 112 is a complementary metal oxide semiconductor image sensor (CIS). The imaging element 112 may be a non-CIS image sensor. For example, the imaging element 112 may be a charge coupled device (CCD) image sensor.

The support member 113 supports the lens 111.

### 1.3 Group of Pixels

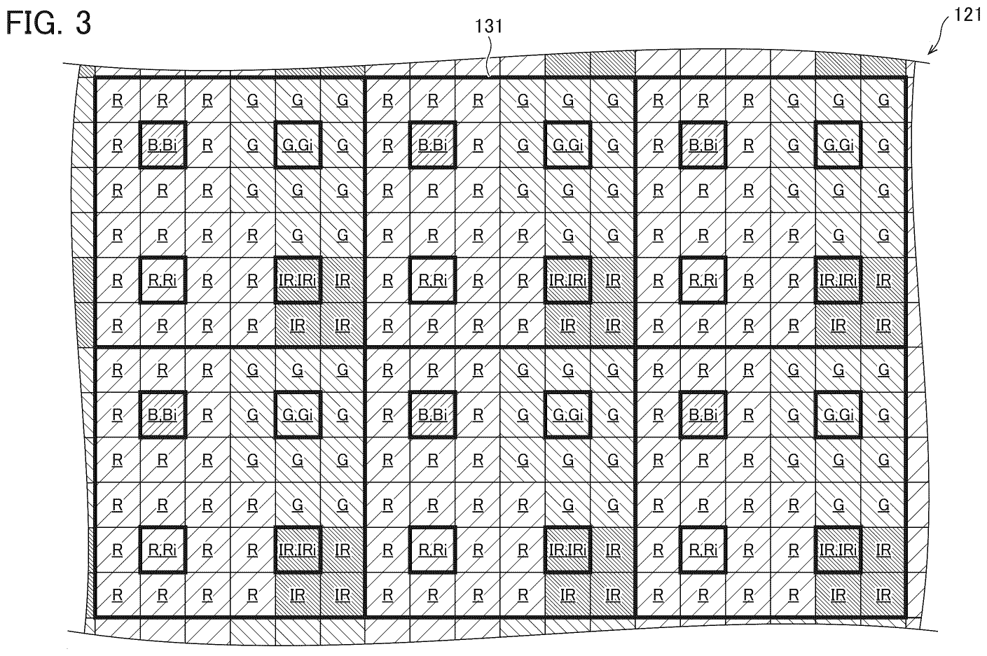

FIG. 3 is a schematic plan view of a group of pixels included in the measuring instrument in accordance with Embodiment 1.

Referring to FIG. 3, the group of pixels 121 includes a group of red pixels R, a group of green pixels G, a group of blue pixels B, and a group of infrared light pixels IR.

Red pixels R, green pixels G, blue pixels B, and infrared light pixels IR included respectively in the group of red pixels R, the group of green pixels G, the group of blue pixels B, and the group of infrared light pixels IR are arranged in a cyclic pattern. Therefore, the imaging element 112 includes a plurality of basic units 131. The plurality of basic units 131 are arranged in a matrix in a light-receiving face of the imaging element 112. The plurality of basic units 131 may be arranged in a non-matrix pattern. Each basic unit 131 includes red pixels R, green pixels G, a blue pixel B, and infrared light pixels IR.

Each of the group of red pixels R, the group of green pixels G, the group of blue pixels B, and the group of infrared light pixels IR includes a group of red pixels Ri for image data, a group of green pixels Gi for image data, a group of blue pixels Bi for image data, and a group of infrared light pixels IRi for image data respectively. Each of the plurality of basic units 131 includes one red pixel Ri for image data, one green pixel Gi for image data, one blue pixel Bi for image data, and one infrared light pixel IRi for image data included respectively in the group of red pixels Ri for image data, the group of green pixels Gi for image data, the group of blue pixels Bi for image data, and the group of infrared light pixels IRi for image data.

### 1.4 Acquisition of Volume Pulse Wave and Image Data

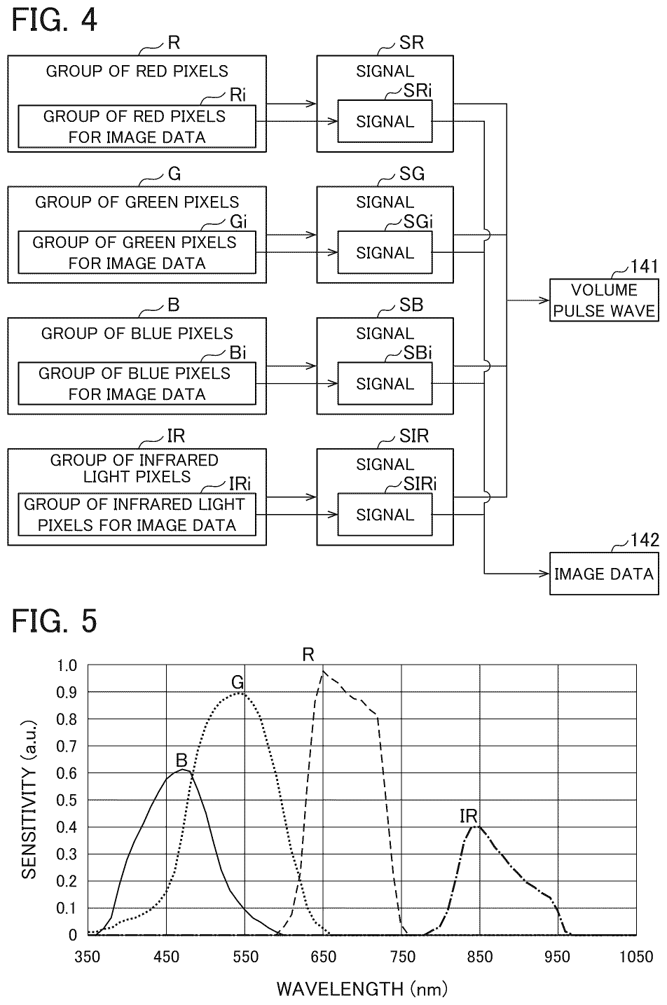

FIG. 4 is a diagram illustrating the content of a process of acquiring a volume pulse wave and image data performed by a processing unit included in the measuring instrument in accordance with Embodiment 1.

Referring to FIG. 4, the processing unit 102 acquires a volume pulse wave 141 by processing signals SR, SG, SB, and SIR respectively representing the quantities of the light received by the group of red pixels R, the group of green pixels G, the group of blue pixels B, and the group of infrared light pixels IR. In doing so, the processing unit 102 specifies, as a reference, the signal SR representing the quantity of the light received by the group of red pixels R that exhibit a peak sensitivity wavelength that falls in a wavelength range where oxidized hemoglobin has a small absorption coefficient. The processing unit 102, for example, performs a process of subtracting the signal SR from the signal SG representing the quantity of the light received by the group of green pixels G that exhibit a peak sensitivity wavelength that falls in a wavelength range where oxidized hemoglobin has a large absorption coefficient. In other words, the processing unit 102 performs a process of subtracting the signal SR, which contains no large volume pulse wave component, from the signal SG, which contains a large volume pulse wave component. Hence, the processing unit 102 restrains the volume pulse wave 141 from being affected by, for example, body movement artifacts of the living body LB and variations of environmental light.

In addition, the processing unit 102 acquires image data 142 representing a color image from signals SRi, SGi, SBi, and SIRi respectively representing the quantities of the light received by the group of red pixels Ri for image data, the group of green pixels Gi for image data, the group of blue pixels Bi for image data, and the group of infrared light pixels IRi for image data.

The processing unit 102 detects a surrounding environment of the measuring instrument 1 from the acquired image data 142. In addition, the processing unit 102 detects, for example, the area of an image of the skin of the living body LB and movements of an image of the living body LB from the acquired image data 142. In addition, the processing unit 102 removes the influence of body movements of the living body LB from the volume pulse wave 141 by using detected movements of the image of the living body LB.

Each of the plurality of basic units 131 includes one red pixel Ri for image data, one green pixel Gi for image data, one blue pixel Bi for image data, and one infrared light pixel IRi for image data. The processing unit 102 acquires respective pixel values of the plurality of basic units 131 from signals representing the quantities of the light received by the red pixel Ri for image data, the green pixel Gi for image data, the blue pixel Bi for image data, and the infrared light pixel IRi for image data included in each of the plurality of basic units 131. Each of the plurality of basic units 131 may include two or more red pixels Ri for image data, two or more green pixels Gi for image data, two or more blue pixels Bi for image data, and two or more infrared light pixels IRi for image data. The processing unit 102 may acquire respective pixel values of the plurality of basic units 131 from signals representing the quantities of the light received by the two or more red pixels Ri for image data, the two or more green pixels Gi for image data, the two or more blue pixels Bi for image data, and the two or more infrared light pixels IRi for image data included in each of the plurality of basic units 131. When this is the case, the processing unit 102 may perform a process of averaging the pixel values representing the quantities of the light received by the two or more red pixels Ri for image data, may perform a process of averaging the pixel values representing the quantities of the light received by the two or more green pixels Gi for image data, may perform a process of averaging the pixel values representing the quantities of the light received by the two or more blue pixels Bi for image data, and may perform a process of averaging the pixel values representing the quantities of the light received by the two or more infrared light pixels IRi for image data.

### 1.5 Spectral Sensitivities of Pixels

FIG. 5 is a graph representing an example of the spectral sensitivities of pixels included in the measuring instrument in accordance with Embodiment 1.

FIG. 5 shows wavelengths on the horizontal axis and sensitivities on the vertical axis.

Referring to FIG. 5, the red pixel R exhibits high sensitivity to red light and has a peak sensitivity wavelength of approximately 650 nm. The green pixel G exhibits high sensitivity to green light and has a peak sensitivity wavelength of approximately 540 nm. The blue pixel B exhibits high sensitivity to blue light and has a peak sensitivity wavelength of approximately 470 nm. The infrared light pixel IR exhibits high sensitivity to infrared light and has a peak sensitivity wavelength of approximately 850 nm.

The signal representing the quantity of the light received by a group of first pixels that exhibit a peak sensitivity wavelength of greater than or equal to 620 nm and less than or equal to 740 nm contains no large volume pulse wave component. The signal representing the quantity of the light received by a group of second pixels that exhibit a peak sensitivity wavelength of either less than or equal to 600 nm or greater than or equal to 760 nm contains a large volume pulse wave component. Therefore, the processing unit 102 acquires the volume pulse wave 141 by processing the signals SR, SG, SB, and SIR respectively representing the quantities of the light received by the group of red pixels R included in the group of first pixels, the group of green pixels G included in the group of second pixels, the group of blue pixels B included in the group of second pixels, and the group of infrared light pixels IR included in the group of second pixels. In doing so, the processing unit 102 specifies the signal SR as a reference.

A group of red pixels included in a typical image-capturing imaging element exhibits a spectral sensitivity that is suited to human visual sensitivity characteristics. Therefore, this group of red pixels exhibits a peak sensitivity wavelength of approximately 600 nm. However, the absorption coefficient of oxidized hemoglobin is not sufficiently small at the wavelength of approximately 600 nm. Therefore, the volume pulse wave component contained in the signal representing the quantity of the light received by the group of red pixels included in the typical image-capturing imaging element is not sufficiently small. Therefore, when this signal is specified as a reference, and for example, a process is performed of subtracting the signal representing the quantity of the light received by the group of red pixels from the signal representing the quantity of the light received by the group of green pixels, the volume pulse wave component is cancelled out. Therefore, the volume pulse wave acquired through this process has a small amplitude. Therefore, it becomes difficult to calculate, for example, a heart rate, a blood pressure, a respiration rate, and a blood oxygen saturation level from a volume pulse wave with high precision, in particular, to calculate biological information other than the heart rate with high precision.

In contrast, the group of red pixels R included in the imaging element 112 exhibits a peak sensitivity wavelength of greater than or equal to 620 nm and less than or equal to 740 nm. At wavelengths of greater than or equal to 620 nm and less than or equal to 740 nm, the absorption coefficient of oxidized hemoglobin is sufficiently small. Therefore, the volume pulse wave component contained in the signal SR representing the quantity of the light received by the group of red pixels R included in the imaging element 112 is sufficiently small. Therefore, when the signal SR is specified as a reference, and for example, a process is performed of subtracting the signal SR from the signal SG representing the quantity of the light received by the group of green pixels G, the volume pulse wave component is not cancelled out. Therefore, the volume pulse wave acquired through this process has no small amplitude. Therefore, it becomes easy to calculate, for example, a heart rate, a blood pressure, a respiration rate, and a blood oxygen saturation level from a volume pulse wave with high precision, in particular, to calculate biological information other than the heart rate with high precision.

In the imaging element 112, a color filter included in the group of red pixels R is a color filter that selectively transmits light with wavelengths of greater than or equal to 620 nm and less than or equal to 740 nm. Hence, the sensitivity wavelength range to which the group of red pixels R is sensitive is limited to a range of greater than or equal to 620 nm and less than or equal to 740 nm. Hence, the volume pulse wave component contained in the signal SR representing the quantity of the light received by the group of red pixels R can be reduced.

However, when the sensitivity wavelength range to which the group of red pixels R is sensitive is limited to a range of greater than or equal to 620 nm and less than or equal to 740 nm, the group of red pixels R becomes less sensitive. Therefore, the signal SR representing the quantity of the light received by the group of red pixels R has a low signal-to-noise ratio. Therefore, the acquired volume pulse wave 141 has a low signal-to-noise ratio.

In addition, for example, under sunlight and light emitted by a white light-emitting diode, green components have the largest quantity of light, and red components have a smaller quantity of light than do green components. Therefore, when the living body LB is illuminated by, for example, sunlight or light emitted by a white light-emitting diode, the quantity of the light received by the group of red pixels R is insufficient. Therefore, the signal SR representing the quantity of the light received by the group of red pixels R has a low signal-to-noise ratio. Therefore, the acquired volume pulse wave 141 has a low signal-to-noise ratio.

### 1.6 Light-Receiving Area of Group of Pixels

Referring to FIG. 3, in the imaging element 112, the light-receiving areas of a plurality of pixels included in the group of pixels 121 are rendered equal to each other. However, in the imaging element 112, since the group of red pixels R has a low sensitivity and/or the quantity of the light received by the group of red pixels R is insufficient, the number of pixels included in the group of red pixels R is rendered larger than the number of pixels included in the group of green pixels G, the number of pixels included in the group of blue pixels B, and the number of pixels included in the group of infrared light pixels IR, to compensate for the low signal-to-noise ratio of the signal SR representing the quantity of the light received by the group of red pixels R. Hence, the sum of the light-receiving areas of the group of red pixels R is rendered larger than the sum of the light-receiving areas of the group of green pixels G, the sum of the light-receiving areas of the group of blue pixels B, and the sum of the light-receiving areas of the group of infrared light pixels IR. This particular approach enables increasing the quantity of the light received by the group of red pixels R to increase the signal-to-noise ratio of the signal SR representing the quantity of the light received by the group of red pixels R.

In a typical image-capturing imaging element, the number of pixels included in the group of green pixels that exhibit high sensitivity to green light to which human visual sensitivity is highest is rendered larger than the number of pixels included in each group of pixels of the other colors. Therefore, in a typical image-capturing imaging element, the total light-receiving area of the group of green pixels is rendered larger than the total light-receiving area of each group of pixels of the other colors.

In contrast, in the imaging element 112, the number of pixels included in the group of red pixels R is rendered larger than the number of pixels included in each group of pixels of the other colors. Therefore, the total light-receiving area of the group of red pixels R is rendered larger than the total light-receiving area of each group of pixels of the other colors. This particular approach can restrain the influence of noise on the measurement of biological information.

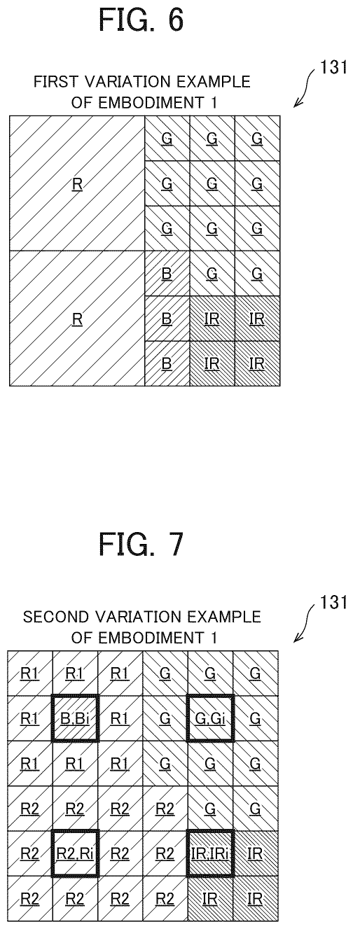

FIG. 6 is a schematic plan view of a basic unit included in a measuring instrument in accordance with a first variation example of Embodiment 1.

Referring to FIG. 6, in the first variation example of Embodiment 1, the pixel area of each infrared light pixel included in the group of red pixels R is rendered larger than the pixel area of each green pixel included in the group of green pixels G, the pixel area of each blue pixel included in the group of blue pixels B, and the pixel area of each infrared light pixel included in the group of infrared light pixels IR. Hence, the sum of the light-receiving areas of the group of red pixels R is rendered larger than the sum of the light-receiving areas of the group of green pixels G, the sum of the light-receiving areas of the group of blue pixels B, and the sum of the light-receiving areas of the group of infrared light pixels IR.

### 1.7 Presence of Both Group of Long-Wavelength-End Pixels and Group of Short-Wavelength-End Pixels

The group of second pixels that exhibit a peak sensitivity wavelength of either less than or equal to 600 nm or greater than or equal to 760 nm includes a group of short-wavelength-end pixels composed of the group of green pixels G and the group of blue pixels B, the short-wavelength-end pixels exhibiting a peak sensitivity wavelength of less than or equal to 600 nm and a group of long-wavelength-end pixels composed of the group of infrared light pixels IR, the long-wavelength-end pixels exhibiting a peak sensitivity wavelength of greater than or equal to 760 nm. This inclusion in the group of second pixels of both the group of short-wavelength-end pixels that exhibit high sensitivity to visible light and the group of long-wavelength-end pixels that exhibit high sensitivity to infrared light enables increasing the types of light sources that can be used as a light source for illuminating the living body LB.

### 1.8 Arrangement of Pixels of Same Color

Referring to FIG. 3, each of the plurality of basic units 131 includes two or more pixels that are included in the group of pixels 121. The two or more pixels include a plurality of red pixels R, a plurality of green pixels G, one blue pixel B, and a plurality of infrared light pixels IR included respectively in the group of red pixels R, the group of green pixels G, the group of blue pixels B, and the group of infrared light pixels IR.

The plurality of red pixels R are disposed in a single cluster. In other words, each red pixel R in the plurality of red pixels R is adjacent to any of the remaining red pixels R in the plurality of red pixels R. Likewise the plurality of green pixels G are also disposed in a single cluster. The plurality of infrared light pixels IR are also disposed in a single cluster.

When mutually adjacent pixels are of different colors, the mutually adjacent color filters included respectively in the mutually adjacent pixels are also of different colors. In addition, in many cases, the boundaries of mutually adjacent color filters overlap. Therefore, when mutually adjacent pixels are of different colors, the boundaries of color filters of different colors overlap. This can cause a decrease in the sensitivity of the mutually adjacent pixels.

However, when the plurality of pixels of the same color included in each of the plurality of basic units 131 are disposed in a single cluster as described above, these boundaries that can cause a decrease in the sensitivity can be reduced.

### 1.9 Colors of Group of Pixels in Group of First Pixels and Group of Second Pixels

In Embodiment 1, the group of first pixels that exhibit a peak sensitivity wavelength of greater than or equal to 620 nm and less than or equal to 740 nm includes a group of pixels of one color composed of the group of red pixels R. In addition, the group of second pixels that exhibit a peak sensitivity wavelength of either less than or equal to 600 nm or greater than or equal to 760 nm includes groups of pixels of three colors composed of the group of green pixels G, the group of blue pixels B, and the group of infrared light pixels IR. In addition, the group of short-wavelength-end pixels included in the group of second pixels, the short-wavelength-end pixels exhibiting a peak sensitivity wavelength of less than or equal to 600 nm, includes groups of pixels of two colors composed of the group of green pixels G and the group of blue pixels B. In addition, the group of long-wavelength-end pixels included in the group of second pixels, the long-wavelength-end pixels exhibiting a peak sensitivity wavelength of greater than or equal to 760 nm, includes a group of pixels of one color composed of the group of infrared light pixels IR. However, the number of the colors of the group(s) of pixels included in the group of first pixels, the number of the colors of the group(s) of pixels included in the group of second pixels, the number of the colors of the group(s) of pixels included in the group of short-wavelength-end pixels, and the number of the colors of the group(s) of pixels included in the group of long-wavelength-end pixels may be increased or decreased. Such examples will be described in the following.

FIG. 7 is a schematic plan view of a basic unit included in a measuring instrument in accordance with a second variation example of Embodiment 1.

Referring to FIG. 7, in the second variation example of Embodiment 1, the group of first pixels that exhibit a peak sensitivity wavelength of greater than or equal to 620 nm and less than or equal to 740 nm includes groups of pixels of two colors composed of a group of first infrared light pixels IR1 and a group of second infrared light pixels IR2. The group of first infrared light pixels IR1 and the group of second infrared light pixels IR2 exhibit mutually different spectral sensitivities. The group of first pixels may include groups of pixels of three or more colors.

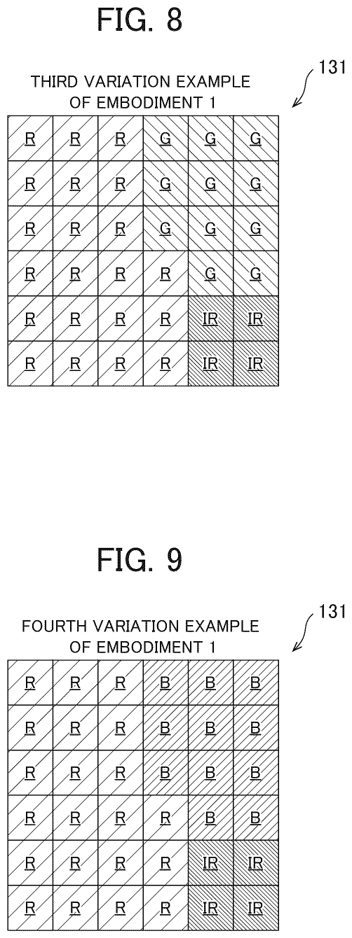

FIG. 8 is a schematic plan view of a basic unit included in a measuring instrument in accordance with a third variation example of Embodiment 1.

Referring to FIG. 8, in the third variation example of Embodiment 1, the group of short-wavelength-end pixels that exhibit a peak sensitivity wavelength of less than or equal to 600 nm does not include the group of blue pixels B and includes only a group of pixels of one color composed of the group of green pixels G.

FIG. 9 is a schematic plan view of a basic unit included in a measuring instrument in accordance with a fourth variation example of Embodiment 1.

Referring to FIG. 9, in the fourth variation example of Embodiment 1, the group of short-wavelength-end pixels that exhibit a peak sensitivity wavelength of less than or equal to 600 nm does not include the group of green pixels G and includes only a group of pixels of one color composed of the group of blue pixels B.

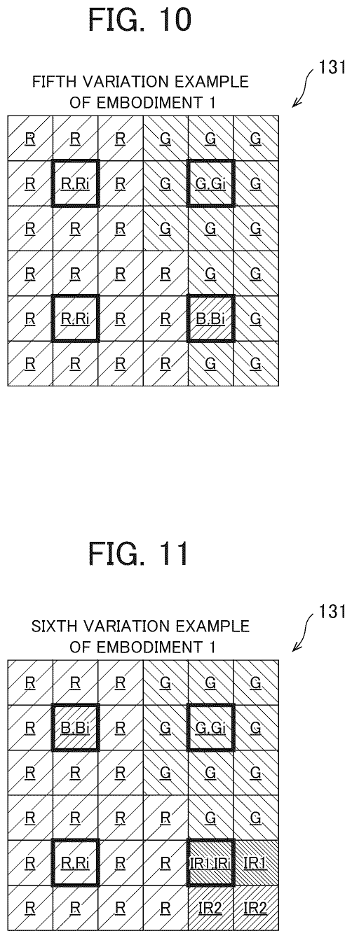

FIG. 10 is a schematic plan view of a basic unit included in a measuring instrument in accordance with a fifth variation example of Embodiment 1.

Referring to FIG. 10, in the fifth variation example of Embodiment 1, the group of second pixels that exhibit a peak sensitivity wavelength of either less than or equal to 600 nm or greater than or equal to 760 nm includes the group of short-wavelength-end pixels composed of the group of green pixels G and the group of blue pixels B, the short-wavelength-end pixels exhibiting a peak sensitivity wavelength of less than or equal to 600 nm, but does not include the group of long-wavelength-end pixels that exhibit a peak sensitivity wavelength of greater than or equal to 760 nm. When this is the case, the group of second pixels entirely exhibits a peak sensitivity wavelength of less than or equal to 600 nm.

FIG. 11 is a schematic plan view of a basic unit included in a measuring instrument in accordance with a sixth variation example of Embodiment 1.

Referring to FIG. 11, in the sixth variation example of Embodiment 1, the group of long-wavelength-end pixels that exhibit a peak sensitivity wavelength of greater than or equal to 760 nm includes groups of pixels of two colors composed of the group of first infrared light pixels IR1 and the group of second infrared light pixels IR2. The group of first infrared light pixels IR1 and the group of second infrared light pixels IR2 exhibit mutually different spectral sensitivities.

### 1.10 Bayer Pattern

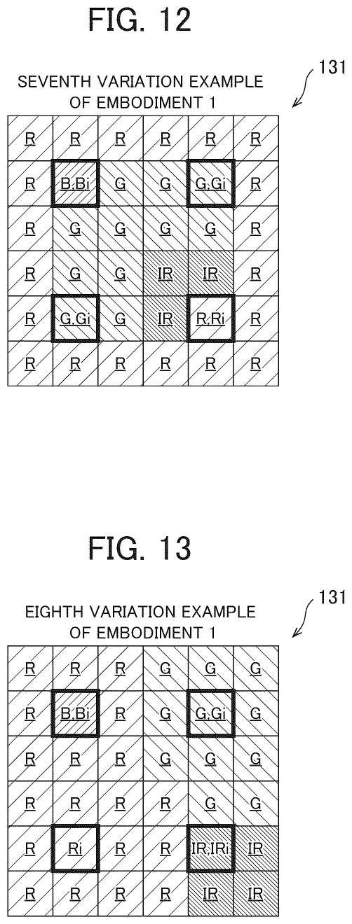

FIG. 12 is a schematic plan view of a basic unit included in a measuring instrument in accordance with a seventh variation example of Embodiment 1.

Referring to FIG. 12, in the seventh variation example of Embodiment 1, the group of red pixels Ri for image data, the group of green pixels Gi for image data, and the group of blue pixels Bi for image data are arranged in a Bayer pattern. In addition, the processing unit 102 performs Bayer interpolation in acquiring the image data 142 from the signals SRi, SGi, and SBi respectively representing the quantities of the light received by the group of red pixels Ri for image data, the group of green pixels Gi for image data, and the group of blue pixels Bi for image data.

When a group of red pixels Ri for image data, the group of green pixels Gi for image data, and the group of blue pixels Bi for image data are not arranged in a Bayer pattern, the resolution of the image data 142 acquired from the signals SRi, SGi, and SBi respectively representing the quantities of the light received by the group of red pixels Ri for image data, the group of green pixels Gi for image data, and the group of blue pixels Bi for image data decreases in accordance with the cycle period of red pixels Ri for image data, the green pixels Gi for image data, and the blue pixels Bi for image data. As an example, when the red pixels Ri for image data, the green pixels Gi for image data, and the blue pixels Bi for image data have a cycle period 6 times the cycle period of pixels in each of the horizontal and vertical directions as shown in FIG. 3, the image data 142 has a resolution 1/36 times the resolution of the group of pixels 121.

In contrast, when the group of red pixels Ri for image data, the group of green pixels Gi for image data, and the group of blue pixels Bi for image data are arranged in a Bayer pattern, although the resolution of the image data 142 acquired from the signals SRi, SGi, and SBi respectively representing the quantities of the light received by the group of red pixels Ri for image data, the group of green pixels Gi for image data, and the group of blue pixels Bi for image data decreases in accordance with the cycle period of the red pixels Ri for image data, the green pixels Gi for image data, and the blue pixels Bi for image data, the decrease is restrained by performing Bayer interpolation. As an example, when the red pixels Ri for image data, the green pixels Gi for image data, and the blue pixels Bi for image data have a cycle period 6 times the cycle period of pixels in each of the horizontal and vertical directions as shown in FIG. 11, the image data 142 has a resolution 1/9 times the resolution of the group of pixels 121.

### 1.11 Separating Infrared Light Pixels for Biological Information Measurement and Infrared Light Pixels for Image Data

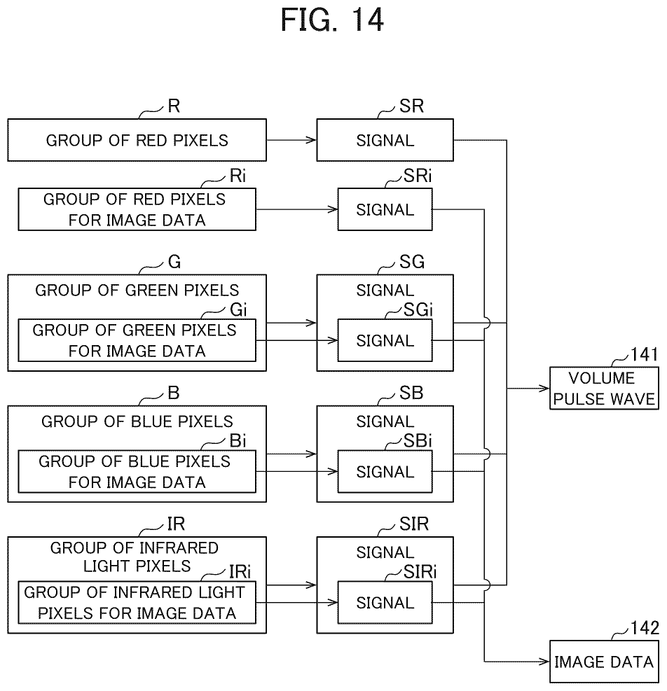

FIG. 13 is a schematic plan view of a basic unit included in a measuring instrument in accordance with an eighth variation example of Embodiment 1.

Referring to FIG. 13, in the eighth variation example of Embodiment 1, the imaging element 112 includes the group of red pixels Ri for image data that exhibit a spectral sensitivity that differs from the spectral sensitivity of the group of red pixels R. The group of red pixels Ri for image data, similarly to a group of red pixels included in a typical image-capturing imaging element, exhibits a spectral sensitivity that is suited to human visual sensitivity characteristics and has a peak sensitivity wavelength of approximately 600 nm.

FIG. 14 is a diagram illustrating the content of a process of acquiring a volume pulse wave and image data performed by a processing unit included in the measuring instrument in accordance with the eighth variation example of Embodiment 1.

Referring to FIG. 14, the processing unit 102 acquires the volume pulse wave 141 by processing the signals SR, SG, SB, and SIR respectively representing the quantities of the light received by the group of red pixels R, the group of green pixels G, the group of blue pixels B, and the group of infrared light pixels IR.

In addition, the processing unit 102 acquires the image data 142 representing a color image from the signals SRi, SGi, SBi, and SIRi respectively representing the quantities of the light received by the group of red pixels Ri for image data, the group of green pixels Gi for image data, the group of blue pixels Bi for image data, and the group of infrared light pixels IRi for image data. It should be noted however that the signal SRi representing the quantity of the light received by the group of red pixels Ri for image data is not a signal included in the signal SR representing the quantity of the light received by the group of red pixels R for volume pulse wave acquisition and is a signal different from the signal SR.

While there have been described what are at present considered to be certain embodiments of the invention, it will be understood that various modifications may be made thereto, and it is intended that the appended claims cover all such modifications as fall within the true spirit and scope of the invention.

What is claimed is:

1. A measuring instrument comprising:
a camera that captures an image of a living body at a distance from the living body, the camera comprising an imaging element comprising:
a group of first pixels that exhibit a peak sensitivity wavelength of greater than or equal to 620 nm and less than or equal to 740 nm; and
a group of second pixels including a group of pixels of at least one color, the second pixels exhibiting a peak sensitivity wavelength of either less than or equal to 600 nm or greater than or equal to 760 nm, the group of first pixels having a total light-receiving area that is larger than a total light-receiving area of a group of pixels of each of the at least one color; and
a processing unit that acquires biological information of the living body by processing a signal representing quantities of light received by the group of first pixels and the group of second pixels.

2. The measuring instrument according to claim 1, wherein the group of second pixels includes a group of short-wavelength-end pixels that exhibit a peak sensitivity wavelength of less than or equal to 600 nm and a group of long-wavelength-end pixels that exhibit a peak sensitivity wavelength of greater than or equal to 760 nm.

3. The measuring instrument according to claim 1, wherein the group of second pixels exhibits a peak sensitivity wavelength of less than or equal to 600 nm.

4. The measuring instrument according to claim 1, wherein the group of first pixels includes groups of pixels of two or more colors that exhibit mutually different spectral sensitivities.

5. The measuring instrument according to claim 1, wherein the group of second pixels includes groups of pixels of two or more colors that exhibit mutually different spectral sensitivities.

6. The measuring instrument according to claim 1, wherein
the imaging element includes a plurality of basic units arranged in a pattern,
each of the plurality of basic units includes two or more pixels, and
pixels of a same color included in the two or more pixels are disposed in a single cluster.

7. The measuring instrument according to claim 1, wherein
the imaging element includes a plurality of basic units arranged in a pattern,
the group of first pixels includes a group of red pixels,
the group of second pixels includes a group of green pixels and a group of blue pixels,
each of the plurality of basic units includes a red pixel, a green pixel, and a blue pixel included respectively in the group of red pixels, the group of green pixels, and the group of blue pixels, and
the processing unit acquires image data representing a color image from a signal representing quantities of light received by the group of red pixels, the group of green pixels, and the group of blue pixels.

8. The measuring instrument according to claim 1, wherein
the imaging element includes a plurality of basic units arranged in a pattern,
the group of first pixels includes a group of red pixels,
the group of second pixels includes a group of green pixels and a group of blue pixels, and
each of the plurality of basic units includes a red pixel, a green pixel, and a blue pixel included respectively in the group of red pixels, the group of green pixels, and the group of blue pixels and arranged in a Bayer pattern.

9. The measuring instrument according to claim 1, wherein
the group of first pixels includes a group of first red pixels,
the imaging element includes a group of second red pixels that exhibit a different spectral sensitivity from a spectral sensitivity of the group of first red pixels, and
the processing unit acquires the biological information by processing a signal representing a quantity of light received by the group of first red pixels and acquires image data representing a color image from a signal representing a quantity of light received by the group of second red pixels.
